(19) United States
(12) Patent Application Publication
(10) Pub. No.: US 2013/0204101 A1
(43) Pub. Date: Aug. 8, 2013

(54) Apparatus and Method for Detecting Skin Cancer Using THz Radiation

(71) Applicants: Axel Rumberg, Karlsruhe (DE); Michael Thiel, Leonberg (DE); Ulrich Kallmann, Tuebingen (DE)

(72) Inventors: Axel Rumberg, Karlsruhe (DE); Michael Thiel, Leonberg (DE); Ulrich Kallmann, Tuebingen (DE)

(73) Assignee: ROBERT BOSCH GMBH, Stuttgart (DE)

(21) Appl. No.: 13/636,171

(22) PCT Filed: Jan. 26, 2011
(86) PCT No.: PCT/EP11/51011

§ 371 Date: Nov. 26, 2012

(30) Foreign Application Priority Data
Mar. 25, 2010 (DE) 102010003239.5

(51) Int. Cl. A61B 5/00

(52) U.S. Cl. CPC A61B 5/444
USPC 600/306

(57) ABSTRACT

An apparatus is configured to detect skin cancer using THz radiation. A high frequency source generates a high-frequency signal. A power splitter divides the high-frequency signal between a transmission branch and a reception branch. A first frequency multiplier multiplies the frequency of the high-frequency signal. A transmission antenna emits the multiplied high-frequency signal as THz radiation. A frequency generator generates a low-frequency signal. A mixer mixes the high-frequency and low-frequency signals to generate a mixed-frequency signal. A second frequency multiplier multiplies the mixed-frequency signal. A reception antenna device receives the THz radiation and generates a THz signal. The mixing device mixes the THz signal with multiplied mixed-frequency signal to generate a measurement signal. The evaluation device evaluates the measurement signal.

### PRIOR ART

The invention proceeds from an apparatus and a method for detecting skin cancer using THz radiation.

The use of THz radiation for detecting skin cancer is already known. In the frequency range of 0.1-5 THz, changes in the refractive index and the absorption property of the skin can be analyzed using reflection measurements, with healthy skin cells and cancerous skin cells having differing water content and therefore having a different refractive index and different absorption properties. The deviation to be expected between healthy skin and skin diseased with cancer is approximately 10%. The frequency determines the optical resolution and the penetration depth. Low frequencies around 200 GHz have a resolution of 2.5 mm. Higher frequencies are able to provide higher resolution, but have a lower penetration depth and require more effort during the generation thereof. US 2008/0319321 discloses an imaging examination using THz radiation, with the THz radiation being generated in a dipole antenna by means of femtosecond pulses from a mode-coupled titanium sapphire laser. The THz radiation reflected by a specimen is likewise converted into an electrical signal in a dipole antenna, with said signal then being analyzed. Generating the radiation and detecting the radiation require much effort.

### DISCLOSURE OF THE INVENTION

By contrast, the apparatus and the method for detecting skin cancer using THz radiation according to the present invention as per claim 1 and 14, respectively, are advantageous in that a simple and cost-effective skin-cancer examination is made possible. The apparatus can be designed as a patient instrument, which enables nevi to be observed with accuracy and additionally evaluates the examined nevi on the basis of a water-content analysis. As a result of a relative measurement, the method works independently of the absolute skin moisture. The skin moisture is detected using the THz radiation. The skin region to be examined is illuminated by THz radiation. The reflected radiation is detected and evaluated. As a matter of principle, skin regions which consist of normal (healthy) skin and potentially diseased skin are considered. Using a color display, the examined region is magnified, and possible differences in the skin moisture are represented by an additional discoloring.

The reflected signal is evaluated in respect of magnitude and phase due to the expected differences in refractive index and absorption. The expected differences in magnitude are of the order of 0.5 dB at −8 dB absolute and 1 degree phase difference. The evaluation is carried out using a reception mixer, the local oscillator (LO) of which is slightly detuned in frequency compared to the transmission signal. In terms of magnitude and phase, the output signal of the mixer is proportional to the reflected THz signal and, depending on the frequency offset of the LO, of the order of a few kHz and can be evaluated with cost-effective analog-to-digital (A/D) converters and a data-processing unit.

Exemplary embodiments of the invention will be explained on the basis of the drawings, in which

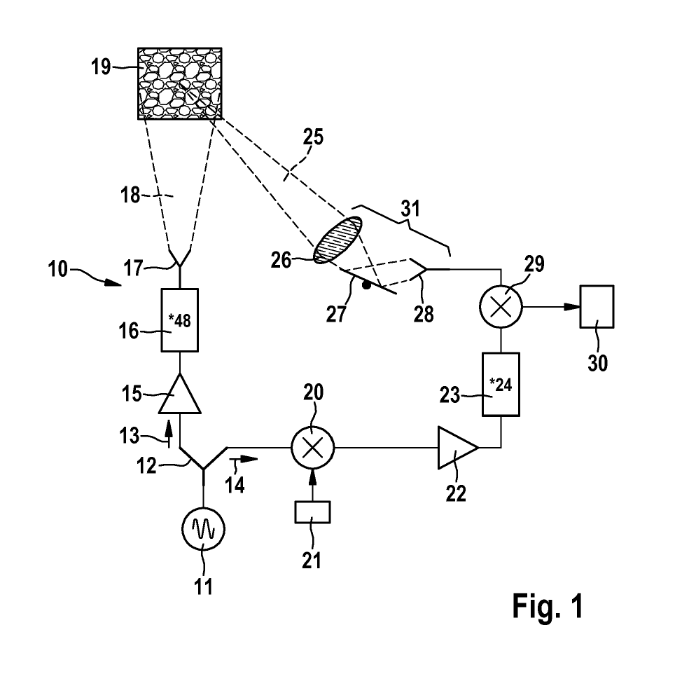

FIG. 1 shows a schematic illustration of an as per a first embodiment of the present invention;

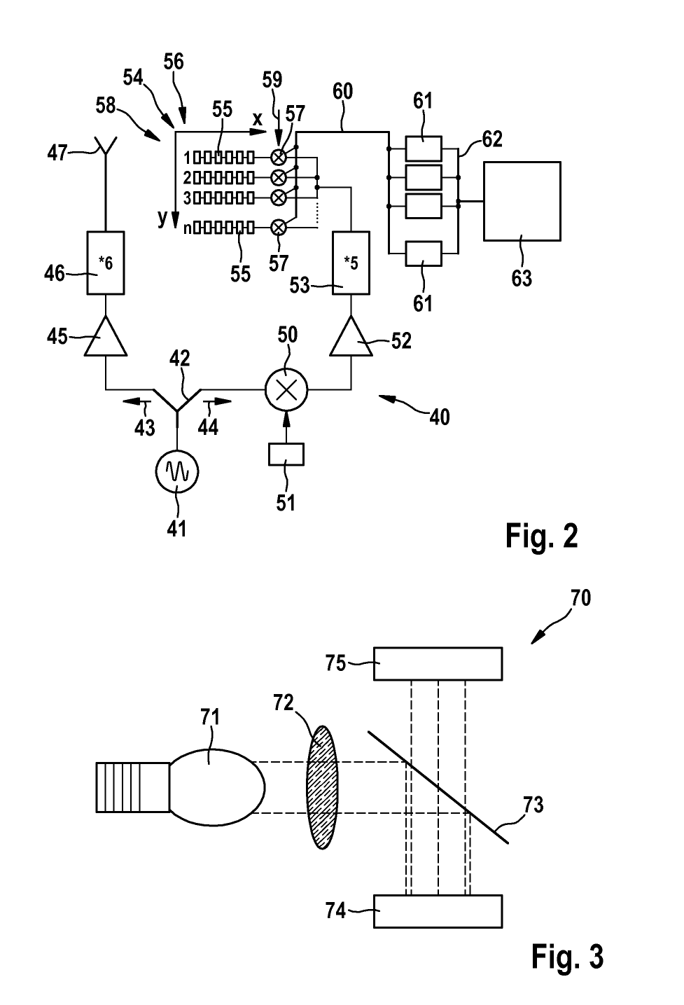

FIG. 2 shows a schematic illustration of an as per a second embodiment of the present invention;

FIG. 3 shows a schematic illustration of an optional complementary apparatus for the first and second embodiment; and

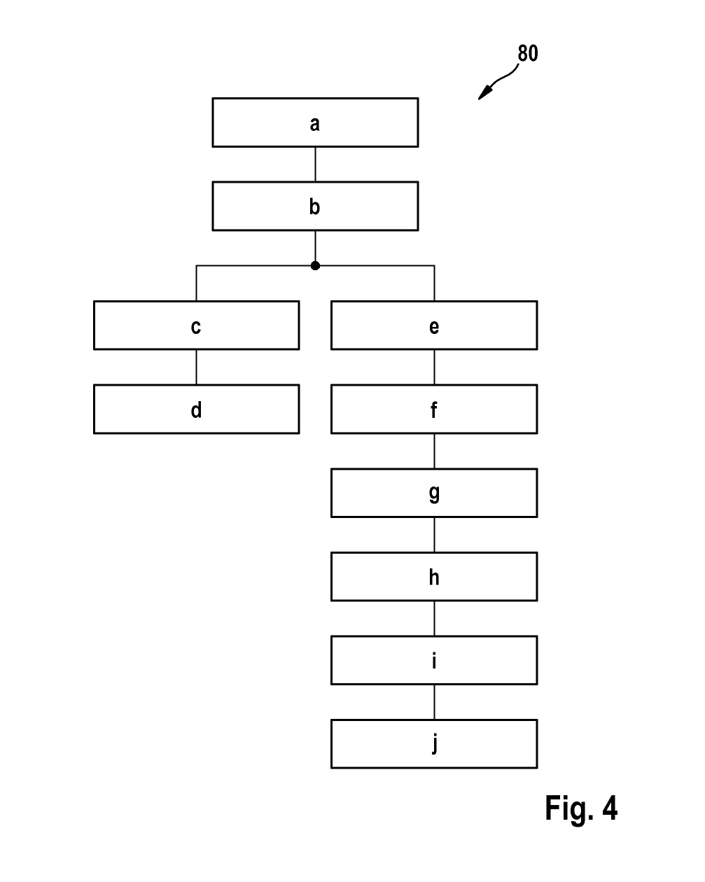

FIG. 4 shows a flowchart of the method as per one embodiment of the present invention.

FIG. 1 illustrates the apparatus 10 for detecting skin cancer using THz radiation, as per a first embodiment of the present invention, and a skin specimen to be examined. The apparatus 10 has a radiofrequency source 11 for generating a radiofrequency signal. The radiofrequency source 11 is connected to a power divider (power splitter 12) for dividing the radiofrequency power between a transmission branch 13 and a reception branch 14. In the transmission branch 13, the power splitter 12 is followed by a first amplifier 15 for amplifying the radiofrequency signal, and this is followed by a first frequency multiplier 16 for multiplying the frequency of the radiofrequency signal. The frequency multiplier 16 is connected to a transmission antenna 17 for emitting the frequency-multiplied radiofrequency signal as transmission THz radiation 18 onto a skin specimen 19.

In the reception branch 14, the power splitter 12 is connected to a mixer 20, which is furthermore connected to a frequency generator or local oscillator 21 for generating a low-frequency signal with a low frequency. The mixer 20 mixes the radiofrequency signal obtained from the power splitter 12 with the low-frequency signal obtained from the local oscillator 21 and generates a reception branch mixed frequency signal with a reception branch mixed frequency. The mixer 20 is connected to a second amplifier 22 for amplifying the reception branch mixed frequency signal, and this is followed by a second frequency multiplier 23 for multiplying the reception branch mixed frequency. The reception THz radiation 25 reflected by the skin specimen 19 is routed through a lens 26 to a horn antenna 28 via a scanner 27. The horn antenna 28 converts the reception THz radiation into an electric reception THz signal and routes the latter to a mixing device 29, which is furthermore connected to the second frequency multiplier 23. The mixing device 29 mixes the reception THz signal with the frequency-multiplied reception branch mixed frequency signal and generates a measurement signal. The mixing device 29 is connected to an evaluation device 30 for evaluating the measurement signal. The lens 26, the scanner 27 and the horn antenna 28 form a reception antenna device 31 for receiving the reception THz radiation and generating a reception THz signal. The reception antenna device 31 is connected to the mixing device 29.

Further details in respect of the first embodiment will now be explained together with the functionality thereof. In respect of the frequencies, this embodiment is presented as a system with frequency multipliers and subharmonic mixer. The frequency multiplication factor N depends strongly on the technology used. In the embodiment presented here, use is made of multipliers with N=48 for the first frequency multiplier 16 and N=24 for the second frequency multiplier 23. The radiofrequency source 11 is implemented at F0=11 GHz and the radiofrequency signal is divided between the transmission branch 13 and the reception branch 14 by means of the power splitter 12. In the transmission branch 13, the signal with the frequency of 11 GHz is amplified to 20 dBm in the first amplifier 15 such that the frequency multiplier 16 is supplied with sufficient power. The frequency multiplier 16 multiplies the radiofrequency of 11 GHz by N=48 to 0.528 THz. This frequency is emitted via the transmission antenna 17, which is embodied as a horn antenna, and the region of the skin to be examined, approximately 1 to 2 cm², is completely illuminated by THz radiation.

In the reception branch and downstream of the power splitter 12, the radiofrequency signal is raised by 50 Hz in terms of its frequency to the reception branch mixed frequency of 11.00000005 GHz with the aid of the mixer 20 embodied as single sideband mixer (SSB-mixer), and said signal is subsequently amplified to approximately 20 dB by the second amplifier 22. In the next stage, the reception branch mixed frequency is multiplied by the factor N=24 to 264.0000012 GHz in the second frequency multiplier 23. This signal is now used as local signal for the following mixing device 29, which is a subharmonic mixer. The reception antenna device 31 is used to geometrically scan the skin field in this embodiment with a scanner 27 as two-axis deflection mirror, wherein the lens is used to image a point of skin onto the horn antenna 28. The reception antenna device 31 routes the reception THz signal to the RF-input of the subharmonic mixer (mixing device 29). The subharmonic mixer makes it possible to mix an RF-signal with double the frequency of the local signal, i.e. 2*264.0000012 GHz-528 GHz=2.4 kHz in this case. The mixer output signal of 2.4 kHz generated thus can be analyzed in respect of magnitude and phase with the aid of a simple analog-to-digital converter within the evaluation device 30. In order to resolve phase differences of 1 degree, a plurality of phase cycles are evaluated, advantageously 10 to 20, such that a jitter of the frequency multipliers over time can be averaged out.

The THz radiation reflected by the specimen is evaluated in the evaluation device 30 in respect of phase and magnitude for a predetermined resolution in the x- and y-directions. In the first embodiment, the desired resolution, e.g. 1 mm², is set by the step size of the scanner motors. All measured values are averaged separately for magnitude and angle. Subsequently, all individual values are separately normalized according to magnitude and phase and the values for magnitude and phase normalized thus are transformed into a scalar value by a suitable geometric addition. The coefficients of the geometric addition are derived from calibration measurements.

FIG. 2 illustrates the apparatus 40 for detecting skin cancer using THz radiation as per a second embodiment of the present invention. The apparatus 40 has a radiofrequency source 41 for generating a radiofrequency signal. The radiofrequency source 41 is connected to a power splitter 42 for dividing the radiofrequency power between a transmission branch 43 and a reception branch 44. In the transmission branch 43, the power divider 42 is followed by a first amplifier 45 for amplifying the radiofrequency signal and said amplifier is followed by a first frequency multiplier 46 for multiplying the frequency of the radiofrequency signal. The frequency multiplier 46 is connected to a transmission antenna 47 for emitting the frequency-multiplied radiofrequency signal as transmission THz radiation onto a skin specimen (not illustrated).

In the reception branch 44, the power splitter 42 is connected to a mixer 50, which is furthermore connected to a frequency generator or local oscillator 51 for generating a low-frequency signal. The mixer 50 mixes the radiofrequency signal obtained from the power splitter 42 with the low-frequency signal obtained from the local oscillator 51 and generates a reception branch mixed frequency signal with a reception branch mixed frequency. The mixer 50 is connected to a second amplifier 52 for amplifying the reception branch mixed frequency signal, said amplifier being followed by a second frequency multiplier 53 for multiplying the reception branch mixed frequency. The reception THz radiation reflected by the skin specimen is received by an antenna array 54 with a number n of antenna rows 55. Together, the antenna rows 55 form a reception antenna device 56. Each antenna row 55 is connected to a mixer 57 assigned thereto. Together, the mixers 57 form a mixing device 59. The mixers 57 are respectively connected to the second frequency multiplier 53 and mix the reception THz signal with the frequency-multiplied reception branch mixed frequency signal and generate an antenna branch measurement signal. The antenna branch measurement signals are fed to a row of analog-to-digital converters 61 via an analog bus 60, with each antenna branch 55 being assigned one analog-to-digital converter 61. The analog-to-digital converters 61 feed a digital output signal to an evaluation unit 63 via a digital bus 62.

Further details in respect of the second embodiment will now be explained in conjunction with the functionality thereof. This embodiment provides a highly integrated solution for implementation with InP, GaAs or SiGe semiconductors. Here, the base frequency is set significantly higher, e.g. at 88 GHz. In this case, the antenna array 54 is embodied as patch array 58. In the x- and y-directions, use is preferably made of between n=6 and n=8 elements, depending on angular resolution. The area of the patch array 58 is 1.5*1.5 mm² in SiGe and 0.9*0.9 mm² on GaAs/InP at an operating frequency of approximately 500 GHz, and hence it can be well integrated. For the second embodiment, 88 GHz were for example selected as generator frequency. In principle, it is also possible to use multiples or splitter frequencies in this case. This must then be taken into account in the factors of the frequency multipliers. The base signal of 88 GHz is completely processed on one or two RF-chips. In the case of two chips, transmitter and receiver are separated. The radiofrequency source 41 with the power splitter 42 and the mixer 50, once again embodied as single sideband mixer (SSB mixer), is implemented either on the transmission chip or on the reception chip. Downstream of the power splitter 42, the transmission signal reaches the first frequency multiplier 46 via the first amplifier 45. The first amplifier 45 ensures a sufficient level for the subsequent first frequency multiplier 46. The first frequency multiplier 46 multiplies the radiofrequency by the factor Ns such that the output signal lies in the vicinity of 500 GHz, here at 528 GHz in the case of a base frequency of 88 GHz with Ns=6. The output signal obtained thus is irradiated on the whole illumination region of the skin specimen via the transmission antenna 47, an external horn antenna or an integrated patch antenna.

In the reception branch, the base signal in this example is mixed with a frequency of 400 Hz from the local oscillator 51 using the SSB mixer 50 in order to form the reception branch mixed frequency of 88 GHz+400 Hz. The signal obtained thus is amplified sufficiently by means of the second amplifier 52 in order to drive the subsequent frequency multiplier in a suitable fashion. The second frequency multiplier 53 multiplies the frequency of the signal by the factor Ns−1, i.e. by a factor of 5 in this case. The output signal is routed to the n subharmonic mixers 57. Alternatively, the multiplication factor in the reception branch is Ns and the n mixers are embodied as simple mixers. The n mixers 57 are each fed by one antenna row 55. The spacing between the antenna rows 55 preferably lies between a quarter wavelength and a whole wavelength. Each antenna row 55 is dimensioned such that in the case of a mid-frequency of F0, in this case 528 GHz, signals perpendicular to the row axis, i.e. with an angle of incidence α=90°, are received with the aperture width β, the latter depending on the number of antenna elements per row. If the base frequency of 88 GHz is modified slightly, then the angle of incidence α changes concurrently. In particular, α decreases for lower frequencies and α increases for higher frequencies, i.e. the reception direction in the x-direction is predetermined by the selection of a frequency shift Δf. Hence, a frequency sweep is used to scan the skin specimen in the x-direction. In the y-direction, the signals from all antenna rows 55 are recorded in parallel by respectively one mixer 57, said signals being mixed down and the mixer output signals being routed via the analog bus 60 to the respective analog-to-digital converters 61. The digital signals downstream of the analog-to-digital converters 61 are subsequently subjected to digital beam forming (DBF) in the evaluation unit 63. With the aid of the DBF method, signals from the y-direction are resolved in accordance with their angle of incidence α with an angular accuracy depending on the number of rows. With the aid of the DBF and the variation of the base frequency, the illuminated skin region can be scanned in the x- and y-directions. The measuring time is only restricted by forming the average to remove the phase jitter on the mixer output signals, said jitter being caused by the many doublers, and by the scanning rate in the x-direction by means of a frequency sweep. The evaluation in the y-direction occurs in parallel in the case of sufficient data-processing computational power for the DBF.

In respect of phase and magnitude for a given resolution, the reflections are evaluated in x- and y-direction in the evaluation unit 63. In the second embodiment, the desired resolution, e.g. 1 mm², is set by the spacing and elements of the antenna array 54. All measured values are averaged separately for magnitude and angle. Subsequently, all individual values are normalized separately according to magnitude and phase, and the values normalized thus for magnitude and phase are transformed into a scalar value by a suitable geometric addition. The coefficients of the geometric addition are derived from calibration measurements.

In the second embodiment, the invention uses a frequency around 0.5 THz, which can be generated and received with a sufficient power in a cost-effective fashion with the aid of InP, GaAs or else modern SiGe RF-processes, and therefore this makes possible instruments which are both compact and cost-effective.

FIG. 3 shows an optional complementary apparatus 70, for the first and second embodiment, for a simple illumination method in the optical spectral range. By means of a lens 72, a lamp 71 illuminates the skin specimen 74 via a beam splitter 73, the image of which skin specimen is recorded by a CCD camera 75. Alternatively, the lamp can be mounted next to the camera and a beam splitter can be dispensed with so as not to impede the beam path of the THz radiation. The evaluation device 30 or 63 generates a superposed image on the basis of the optical image of the CCD camera 75, wherein the colors of the values obtained from the THz measurement are additionally modified by a false color for each cell illuminated by THz radiation such that the points of the skin with a higher water content within the observed scanning region can be uniquely assigned. The examined skin region is illustrated in magnified fashion on a colored standard display of the evaluation device 30 or 63.

When the apparatus from FIG. 1 or FIG. 2 is integrated with the apparatus from FIG. 3 in order to form a complete instrument, the evaluation device 30 or 63 is advantageously embodied as a control and evaluation unit with a display.

Particularly in the case of an embodiment as patient instrument, use can be made of a personal computer in the integration of the apparatus from FIG. 1 or FIG. 2 with the apparatus from FIG. 3, which personal computer assumes the control functions and evaluation functions as part of the evaluation device 30 or 63 and provides the display.

FIG. 4 shows a flowchart 80 of the method as per one embodiment of the present invention. The method for detecting skin cancer using THz radiation by irradiating a specimen with transmission THz radiation and evaluating reception THz radiation originating from the specimen starts with the method step a) of generating a radiofrequency signal. This is subsequently followed by step b) of dividing the radiofrequency signal between the transmission branch and the reception branch. In the transmission branch, this is now followed by method step c) of multiplying the frequency of the radiofrequency signal and method step d) of emitting the frequency-multiplied radiofrequency signal as transmission THz radiation onto a specimen. In the reception branch, method step b) is followed by method step e) of generating a low-frequency signal and method step f) of mixing the radiofrequency signal with the low-frequency signal in order to generate a reception branch mixed frequency signal. This is followed by method step g) of multiplying the reception branch mixed frequency. After method step h) of receiving the reception THz radiation from the specimen and generating a THz signal therefrom, there is method step i) of mixing the THz signal with the frequency-multiplied reception branch mixed frequency signal in order to generate an output signal therefrom, and finally there is method step j) of evaluating the output signal.

Advantageously, a plurality of phase cycles of the reception THz radiation are evaluated for a measurement point.

The specified frequencies and multiplication factors are examples that do not constitute a restriction of the invention but merely explain the design of the interacting components such that the apparatus and the method can be used in the frequency range between 0.1 and 5 THz.

1. An apparatus for detecting skin cancer using THz radiation comprising:
a radiofrequency source configured to generate a radiofrequency signal to be used in a transmission branch and a reception branch;
a power divider configured to divide the radiofrequency signal between the transmission branch and the reception branch;
a first frequency multiplier arranged in the transmission branch and configured to multiply a frequency of the radiofrequency signal;
a transmission antenna arranged in the transmission branch and configured to emit the frequency-multiplied radiofrequency signal as THz radiation;
a frequency generator arranged in the reception branch and configured to generate a low-frequency signal;
a mixer arranged in the reception branch and configured to mix the radiofrequency signal with the low-frequency signal to generate a reception branch mixed frequency signal;
a second frequency multiplier arranged in the reception branch and configured to multiply the reception branch mixed frequency signal;
a reception antenna apparatus arranged in the reception branch and configured to receive the THz radiation and to generate a THz signal;
a mixing device arranged in the reception branch and configured to mix the THz signal with the frequency-multiplied reception branch mixed frequency signal to generate a measurement signal; and
an evaluation device arranged in the reception branch and configured to evaluate the measurement signal.

2. The apparatus of claim 1, wherein the reception antenna device has a lens in a beam path of the THz radiation.

3. The apparatus of claim 2, wherein the reception antenna device has a horn antenna and a scanner in the beam path of the reception THz radiation.

4. The apparatus of claim 3, wherein the scanner has a deflection mirror that is rotatable about two axes.

5. The apparatus of claim 1, wherein:
the second frequency multiplier has a lower multiplication factor than the first frequency multiplier; and
the mixing device has a subharmonic mixer.

6. The apparatus of claim 1, wherein the reception antenna device has an antenna array with a plurality of antenna rows.

7. The apparatus of claim 6, wherein the mixing device has a mixer assigned to each antenna row in the plurality of antenna rows.

8. The apparatus of claim 7, further comprising an analog-to-digital converter assigned to each mixer.

9. The apparatus of claim 7, wherein:
the second frequency multiplier has a lower multiplication factor than the first frequency multiplier; and
the mixers assigned to the antenna rows are subharmonic mixers.

10. The apparatus of claim 7, wherein:
the second frequency multiplier has the same multiplication factor as the first frequency multiplier; and
the mixers assigned to the antenna rows are simple mixers.

11. The apparatus of claim 6, wherein the evaluation device is configured with a digital beam-forming method.

12. The apparatus of claim 1, further comprising a first amplifier arranged in the transmission branch.

13. The apparatus of claim 1, further comprising a second amplifier is arranged in the reception branch.

14. A method for detecting skin cancer using THz radiation comprising:
generating a radiofrequency signal;
dividing the radiofrequency signal between a transmission branch and a reception branch;
multiplying the frequency of the radiofrequency signal in the transmission branch;
emitting the frequency-multiplied radiofrequency signal as transmission THz radiation in the transmission branch onto a specimen so as to generate a reception THz radiation;
generating a low-frequency signal in the reception branch;
mixing the radiofrequency signal with the low-frequency signal to generate a reception branch mixed frequency signal in the reception branch;
multiplying the reception branch mixed frequency signal in the reception branch;
receiving the reception THz radiation from the specimen and generating a THz signal therefrom in the reception branch;
mixing the THz signal with the frequency-multiplied reception branch mixed frequency signal to generate an output signal therefrom in the reception branch; and
evaluating the output signal.

15. The method of claim 14, wherein a plurality of phase cycles of the reception THz radiation are evaluated for a measurement point.
